(19) United States
(12) Patent Application Publication
(10) Pub. No.: US 2026/0144971 A1
(43) Pub. Date: May 28, 2026

(54) A Catheter For Placement In A Ventricular System

(71) Applicant: Rhovica Neuroimaging AG, Bettingen (CH)

(72) Inventors: Thomas Rhomberg, Bludenz (AT); Martin Hlavica, Aarau (CH); Gürkan Yilmaz, Bourg-en-Lavaux (CH); Jonathan Bennes, Pontarlier (FR)

(73) Assignee: Rhovica Neuroimaging AG, Bettingen (CH)

(21) Appl. No.: 19/123,461

(22) PCT Filed: Oct. 24, 2023
(86) PCT No.: PCT/EP2023/079669

§ 371 Date: Apr. 23, 2025

(30) Foreign Application Priority Data
Oct. 28, 2022 (CH) CH001280/2022

(51) Int. Cl. A61M 27/00; A61M 25/01

(52) U.S. Cl. CPC A61M 27/006; A61M 25/0102; A61M 2202/0464; A61M 2205/3375; A61M 2210/0693

(57) ABSTRACT

A catheter, in particular an external ventricular drainage catheter, for placement in a ventricular system, in particular of a human, for drainage of liquid, in particular of cerebrospinal fluid, from a ventricle of the ventricular system, includes a tubular body having an internal drainage lumen running along the tubular body for the drainage of the liquid and a port, arranged in a distal region of the tubular body, the port connecting the drainage lumen with an outside of the tubular body and outside of the catheter for the drainage of the liquid from the ventricle into the drainage lumen. The catheter further includes an ultrasonic sensor, or transducer, for emitting frontal ultrasound waves and detecting the frontal ultrasound waves reflected back to the at least one frontal ultrasonic sensor, in particular for obtaining a sonogram. The frontal ultrasonic sensor is arranged at a distal tip of said tubular body.

### TECHNICAL FIELD

The invention relates to a catheter, in particular an external ventricular drainage catheter, for placement in a ventricular system, in particular of a human, for drainage of liquid, in particular of cerebrospinal fluid (CSF), from a ventricle of the ventricular system, the catheter including a tubular body having an internal drainage lumen running along the tubular body for the drainage of the liquid, and at least one port arranged in a distal region of the tubular body, the at least one port connecting the drainage lumen with an outside of the tubular body and outside of the catheter for the drainage of the liquid from the ventricle into the drainage lumen.

### BACKGROUND ART

Catheters are medical devices that can be inserted in the body to treat diseases or perform a surgical procedure. Such catheters are advantageously made from a thin, flexible tube. As an example, the puncture of a lateral ventricle and placement of a flexible catheter into the ventricular system is a very common procedure performed for a variety of indications including drainage of cerebrospinal fluid (CSF). In such a procedure, usually, a hole is made in the skull with a twist drill or a burr and the catheter with a mandrin, in particular a stylet, inserted in the lumen of the catheter are introduced together, for example through the frontal lobe or posterior occipital lobe of the brain, into the ventricle. Once the catheter is introduced into the ventricle, the mandrin or stylet is removed and the distal tip of the catheter remains in the anterior horn of the ventricle. Cerebrospinal fluid can then be withdrawn from the ventricular system through the catheter.

One example of an arrangement of a catheter pertaining to the technical field initially mentioned and a stylet is described in WO 96/29011 A1 of John Gilbert. In this example, the stylet is a rigid, ultrasonic-fiberoptic stylet providing a longitudinal aperture being packed with fiberoptics and a miniature ultrasonic transducer. This ultrasonic-fiberoptic imaging stylet fits within the catheter and allows viewing through the end of the catheter. Thus, the stylet being situated within the catheter allows indirect and direct real time visualisation through the tip of the catheter. Therefore, the ultrasonic portion of the stylet allows the surgeon to correctly aim the stylet and catheter towards the ventricle by giving the surgeon a 2-dimensional echogram view of the ventricle. This also allows the surgeon to maintain the stylet and catheter in the proper trajectory or path towards the anterior horn of the lateral ventricle as the stylet and catheter are passed through the brain. The fiberoptic portion of the stylet allows the surgeon to directly view the interior of the anterior horn of lateral ventricle once the ventricle is punctured by the stylet and catheter, thus, confirming correct placement.

This known catheter has the disadvantage that once the catheter is positioned in the ventricle and the stylet is removed in order to enable drainage of cerebrospinal fluid, no further monitoring of the proper positioning of the catheter in the ventricle is possible other than by maintaining the catheter fixed externally on the skull.

In principle, the drainage flow of cerebrospinal fluid can additionally be used as an indicator for controlling the proper positioning of the catheter in the ventricle because if the catheter is displaced away from the ventricle, the drainage flow of cerebrospinal fluid is reduced or stopped. However, this indicator is very unreliable because the drainage flow of cerebrospinal fluid is reduced or stopped, too, if the catheter is clogged or if the ventricles are drained empty.

For these reasons, the known catheter cannot be used very reliably and safely for drainage of cerebrospinal fluid.

In the present text, the term “and/or” is sometimes used for linking two features. For example this term is used to link the features A and B in the formulation “A and/or B”. Such a formulation means that at least feature A or feature B is realized. In other words, the formulation includes the option of “A and not B”, the option “B and not A”, as well as the option “A and B”.

In the present text, the terms “distal”, “distally”, “proximal” and “proximally” are used referring to the catheter. Thereby, the distal end of the catheter is the tip of the catheter inserted into the ventricle, while the proximal end of the catheter is the end of the catheter which remains outside of the skull. Correspondingly, a distal end of an element of the catheter is on the side of the respective element facing the distal end of the catheter while the proximal end of the respective element of the catheter is on the side of the respective element facing the proximal end of the catheter. Similarly, a distal region of the catheter is a region in the area of the distal end of the catheter, while a proximal region of the catheter is a region in the area of the proximal end of the catheter.

### SUMMARY OF THE INVENTION

It is the object of the invention to create a catheter, in particular an external ventricular drainage catheter, for placement in a ventricular system, in particular of a human, for drainage of liquid, in particular of cerebrospinal fluid, from a ventricle of the ventricular system, pertaining to the technical field initially mentioned, that enables a reliable and safe use of the catheter for drainage of cerebrospinal fluid.

The solution of the invention is specified by the features of claim 1. According to the invention, the catheter further includes at least one frontal ultrasonic sensor, in particular at least one frontal ultrasonic transducer, for emitting frontal ultrasound waves and detecting the frontal ultrasound waves reflected back to the at least one frontal ultrasonic sensor, in particular for obtaining a sonogram, wherein the at least one frontal ultrasonic sensor is arranged at a distal tip of the tubular body.

According to the invention, the catheter includes a tubular body having an internal drainage lumen running along the tubular body for the drainage of the liquid. This drainage lumen is advantageously formed by the tubular body. For example, the tubular body can be formed by a tube having an internal lumen forming the internal drainage lumen.

Furthermore, according to the invention, the catheter includes at least one port arranged in a distal region of the tubular body. Advantageously, this at least one port is arranged laterally in a sidewall of the tubular body. However, instead of being arranged laterally, the at least one port can as well be arranged in the distal tip of the tubular body facing in a direction along a longitudinal axis of the distal region of the tubular body. Independent of where the at least one port is arranged in the distal region of the tubular body, the at least one port connects the drainage lumen with an outside of the tubular body and outside of the catheter for the drainage of the liquid from the ventricle into the drainage lumen. Thus, each one of the at least one port allows the cerebrospinal fluid to flow through the respective one of the at least one port from the ventricle into the drainage lumen.

According to the invention, the catheter further includes at least one frontal ultrasonic sensor for emitting frontal ultrasound waves and detecting the frontal ultrasound waves reflected back to the at least one frontal ultrasonic sensor, in particular for obtaining a sonogram. Each one of the at least one frontal ultrasonic sensor is advantageously a frontal ultrasonic transducer. An ultrasonic transducer converts an electrical signal into ultrasound waves and converts ultrasound waves into an electrical signal. In order to achieve this conversion, an ultrasonic transducer is operatable in a transmitter mode to convert electrical signals to pressure waves at ultrasound frequencies and thus to ultrasound waves. Furthermore, in order to achieve this conversion, an ultrasonic transducer is operatable in a receiver mode for receiving ultrasound waves and converting the received ultrasound waves into electrical signals. Ultrasonic transducers are typically piezoelectric ultrasonic transducers or capacitive ultrasonic transducers.

From the signals of the emitted frontal ultrasound waves and the detected frontal ultrasound waves reflected back to the at least one frontal ultrasonic sensor, ultrasound images of interfaces, like for example the interface between brain tissue and the cerebrospinal fluid in the ventricle, located in front of the distal tip of the tubular body can be calculated. These ultrasound images are sonograms. Furthermore, instead of obtaining a sonogram or in combination with obtaining a sonogram, from the signals of the emitted frontal ultrasound waves and the detected frontal ultrasound waves reflected back to the at least one frontal ultrasonic sensor, an acoustic signal representing an image of the interfaces, like for example the interface between brain tissue and the cerebrospinal fluid in the ventricle, located in front of the distal tip of the tubular body can be calculated.

Thereby, advantageously, the distal tip of the tubular body is at the same time the distal tip of the catheter. Thus, from the signals of the emitted frontal ultrasound waves and the detected frontal ultrasound waves reflected back to the at least one frontal ultrasonic sensor, sonograms being ultrasound images of interfaces, like for example the interface between brain tissue and the cerebrospinal fluid in the ventricle, located in front of the distal tip of the catheter and/or acoustic signals representing images of the interfaces, like for example the interface between brain tissue and the cerebrospinal fluid in the ventricle, can be calculated.

Due to the at least one frontal ultrasonic sensor for emitting the frontal ultrasound waves and detecting the frontal ultrasound waves reflected back to the at least one frontal ultrasonic sensor a real time visualisation and/or acoustic representation of the interfaces in front of the catheter, like for example the interface between brain tissue and the cerebrospinal fluid in the ventricle in front of the catheter, can be obtained. Consequently, due to the at least one frontal ultrasonic sensor, the navigation of the catheter in the brain and the ventricle is facilitated.

In that the catheter includes the at least one frontal ultrasonic sensor for emitting frontal ultrasound waves and detecting the frontal ultrasound waves reflected back to the at least one frontal ultrasonic sensor, in particular for obtaining a sonograms, the at least one frontal ultrasonic sensor being arranged at the distal tip of the tubular body, an ultrasound image and thus sonogram and/or acoustic representation of the region in front of the distal tip of the tubular body of the catheter can be obtained during drainage of cerebrospinal fluid from the ventricle while the catheter is positioned in the ventricle. Thus, the catheter according to the invention not only enables navigation of the catheter during moving the catheter through the brain to the ventricle but also enables monitoring of the proper positioning of the catheter in the ventricle during drainage of cerebrospinal fluid once the catheter is positioned in the ventricle. Thus, the invention according to the invention enables a reliable and safe use of the catheter for drainage of fluid, in particular of cerebrospinal fluid.

In a preferred variant, the catheter includes exactly one frontal ultrasonic sensor. This has the advantage that the catheter can be produced cheaper while still enabling a reliable and safe use of the catheter for drainage of fluid, in particular of cerebrospinal fluid. In a preferred variant, however, the catheter includes more than one frontal ultrasonic sensor. Such a variant has the advantage that a further improved navigation of the catheter during moving the catheter through the brain to the ventricle and that a further improved monitoring of the proper positioning of the catheter in the ventricle during drainage of cerebrospinal fluid once the catheter is positioned in the ventricle can be achieved.

Advantageously, the tubular body includes an outlet for letting the liquid from the drainage lumen out of the tubular body, the outlet being arranged in a proximal region of the tubular body. This has the advantage that drainage of the liquid from the drainage lumen can be collected in a volume separate from the catheter.

Alternatively, however, the tubular body goes without an outlet for letting the liquid from the drainage lumen out of the tubular bode. In such an alternative, the liquid may for example be collected in the drainage lumen. In this case, the drainage lumen may include a chamber for collecting the liquid, in particular cerebrospinal fluid.

Preferably, one of the at least one frontal ultrasonic sensor is aligned to emit the frontal ultrasound waves from the distal tip of the tubular body away from the tubular body in a direction along a longitudinal axis of the distal region of the tubular body. This has the advantage that the frontal ultrasonic sensor which is aligned to emit the frontal ultrasound waves from the distal tip of the tubular body away from the tubular body in a direction along a longitudinal axis of the distal region of the tubular body allows for obtaining sonograms and thus ultrasound images and/or acoustic representations of interfaces between brain tissue and cerebrospinal fluid in the ventricle in front of the distal tip of the tubular body in the direction along the longitudinal axis of the distal region of the tubular body. Furthermore, this has the advantage that the respective frontal ultrasonic sensor enables detecting these interfaces, thus enabling an improved navigation during insertion of the catheter into the ventricle.

Alternatively, however, each one of the at least one frontal ultrasonic sensor is aligned differently from being aligned to emit the frontal ultrasound waves from the distal tip of the tubular body away from the tubular body in a direction along a longitudinal axis of the distal region of the tubular body.

In a preferred variant, however, the catheter includes more than one frontal ultrasonic sensor, in particular at least three frontal ultrasonic sensors, wherein all the frontal ultrasonic sensors or the frontal ultrasonic sensors with the exception of one of the frontal ultrasonic sensors are aligned to emit the frontal ultrasound waves from the distal tip of the tubular body away from the tubular body in a direction inclined at an angle of at least 5°, particular preferably of at least 10°, to the longitudinal axis of the distal region of the tubular body. In an advantageous variant, the frontal ultrasonic sensors are additionally aligned to emit the frontal ultrasound waves in directions which are inclined to each other by at least 5°, particular advantageously by at least 10°.

This has the advantage that due to the different alignments of the different frontal ultrasonic sensors, an area around the longitudinal axis of the distal region of the tubular body can be scanned by operating the frontal ultrasonic sensors in B-Mode. Thereby, depending on the alignment of the frontal ultrasonic sensors, a plane including the longitudinal axis of the distal region of the tubular body or even a cone around the longitudinal axis of the distal region of the tubular body can be scanned with the frontal ultrasonic sensors.

Advantageously, the at least one frontal ultrasonic sensor is a piezoelectric ultrasonic transducer. Thus, advantageously, the at least one frontal ultrasonic sensor includes a piezoelectric element, in particular a piezoelectric layer. This has the advantage that the at least one frontal ultrasonic sensor can be designed particularly small and thus suitable for being installed in the distal tip of the catheter, in particular at the distal tip of the tubular body.

In a variant, the at least one frontal ultrasonic sensor is a capacitive ultrasonic transducer. In yet another example, the at least one frontal ultrasonic sensor is neither a piezoelectric ultrasonic transducer nor a capacitive ultrasonic transducer. The at least one frontal ultrasonic sensor can also be another sensor than a transducer. For example, the at least one frontal ultrasonic sensor can include a transmitter for emitting the frontal ultrasound waves and a receiver for detecting the frontal ultrasound waves reflected back to the receiver and thus the at least one frontal ultrasonic sensor, in particular for obtaining a sonogram, wherein the receiver is a unit separate from the transmitter. Thereby, the unit of the receiver and the unit of the transmitter can be arranged in one and the same of the at least one frontal ultrasonic sensor.

Advantageously, the at least one frontal ultrasonic sensor is adapted to emit the frontal ultrasound waves at a frequency, in particular a center frequency, in a range from 1 MHz to 20 MHz, particular advantageously from 5 MHz to 12 MHz. This has the advantage that interfaces between brain tissue and cerebrospinal fluid can be detected and visualised in an ultrasound image up to a distance in a range from 4 cm to 10 cm from the at least one frontal ultrasonic sensor and thus in front of the distal tip of the tubular body, in particular in front of the distal tip of the catheter. This has the advantage that the navigation of the catheter during moving the catheter through the brain to the ventricle is facilitated.

Preferably, the at least one frontal ultrasonic sensor is adapted to emit the frontal ultrasound waves with a peak rarefactional pressure in a range from 0 MPa to 7 MPa. This has the advantage that brain injuries during the use of the catheter due to the pressure caused by the frontal ultrasound waves can be avoided. Alternatively, however, the at least one frontal ultrasonic sensor is adapted to emit the frontal ultrasound waves with a peak rarefactional pressure of more than 7 MPa.

Advantageously, the at least one frontal ultrasonic sensor is adapted to emit the frontal ultrasound waves in a pulsed manner with 1 to 100 cycles in one pulse. Thereby, advantageously, the at least one frontal ultrasonic sensor is adapted to emit the frontal ultrasound waves at a pulse repetition frequency in a range from 100 Hz to 20 KHz. However, more than 100 cycles per pulse may be employed, too, and the pulse repetition frequency may be chosen to be smaller than 100 kHz or larger than 20 kHz.

Advantageously, the at least one frontal ultrasonic sensor is adapted to provide in total an acoustic output level I_{SPTA.3} of 94 mW/cm² or less, advantageously I_{SPPA.3} of 190 W/cm² or less, wherein I_{SPTA.3} is the global maximum derated I_{SPTA} intensity value and I_{SPPA.3} is the Mechanical Index (or derated I_{SPPA} intensity) value indicated in Table 3 of “Marketing Clearance of Diagnostic Ultrasound Systems and Transducers”, “Guidance for Industry and Food and Drug Administration Staff” issued on Jun. 27, 2019, of the U.S. Department of Health and Human Services, Food and Drug Administration, Center for Devices and Radiological Health. Alternatively, however, the at least one frontal ultrasonic sensor is adapted to provide in total a higher acoustic output level.

Alternatively, the at least one frontal ultrasonic sensor is adapted to emit the frontal ultrasound waves at a frequency of less than 1 MHz or more than 20 MHz. Advantageously, however, the at least one frontal ultrasonic sensor is adapted to emit the frontal ultrasound waves at a frequency of more than 18 kHz and less than 100 MHz, in particular less than 50 MHz.

Advantageously, the catheter includes at least five ports, particular advantageously at least ten ports, most advantageously at least fifteen ports, arranged in the distal region of the tubular body, the at least five ports, at least ten ports or at least fifteen ports, respectively, connecting the drainage lumen in the distal region of the tubular body with the outside of the tubular body and the outside of the catheter for the drainage of the liquid from the ventricle into the drainage lumen. Thus, each one of the at least five ports, at least ten ports or at least fifteen ports, respectively, allows the fluid to flow through the respective one of the at least five ports, at least ten ports or at least fifteen ports, respectively, from the ventricle into the drainage lumen. This has the advantage that a very efficient drainage of the liquid, in particular of the cerebrospinal fluid, from the ventricle into the drainage lumen is enabled.

Advantageously, the at least five ports, the at least ten ports, or the at least fifteen ports, respectively, are arranged in a port region within in the distal region of the tubular body. Particular advantageously, the port region extends over a length in a range from about 1 cm to about 2 cm, in particular a range from about 1.5 cm to about 2.0 cm, along the tubular body. However, the port region may extend over a length smaller than about 1 cm or over a length larger than about 2 cm. Advantageously, the catheter includes less than five hundred ports arranged in the distal region of the tubular body, the less than five hundred ports connecting the drainage lumen in the distal region of the tubular body with the outside of the tubular body and the outside of the catheter for the drainage of the liquid from the ventricle into the drainage lumen. Alternatively, however, the catheter includes five hundred or more ports arranged in the distal region of the tubular body, the five hundred or more ports connecting the drainage lumen in the distal region of the tubular body with the outside of the tubular body and the outside of the catheter for the drainage of the liquid from the ventricle into the drainage lumen.

Advantageously, the at least five ports, the at least ten ports or the at least fifteen ports, respectively, are arranged laterally in a sidewall of the tubular body. This has the advantage that an efficient drainage of the liquid, in particular of the cerebrospinal fluid, from the ventricle into the drainage lumen is enabled while at the same time, the distal region of the tubular body carrying the at least five ports, the at least ten ports or the at least fifteen ports, respectively, can be designed to provide a high stability and durability.

Alternatively, one or more of the at least five ports, the at least ten ports or the at least fifteen ports, respectively, is not arranged laterally in the sidewall of the tubular body but in the distal tip of the tubular body facing in a direction along a longitudinal axis of the distal region of the tubular body.

Preferably, the at least five ports, at least ten ports or at least fifteen ports, respectively, are distributed around a circumference of the tubular body, in particular to face in all directions around a circumference of the tubular body. Thus, advantageously, in all 360 degrees around the circumference of the tubular body, an opening in the sidewall of the tubular body is provided by at least one of the at least five ports, at least ten ports or at least fifteen ports, respectively. This has the advantage that independent of the orientation with which the catheter is inserted into the ventricle, an optimal drainage of the liquid, in particular of the cerebrospinal fluid, from the ventricle into the drainage lumen is enabled.

Alternatively, however, the at least five ports, at least ten ports or at least fifteen ports, respectively, can be distributed differently in the distal region of the tubular body.

Preferably, the at least one port, the at least five, at least ten or at least fifteen ports, respectively, is/are arranged proximally from the at least one frontal ultrasonic sensor.

This has the advantage that the at least one frontal ultrasonic sensor can be arranged at the distal tip of the tubular body without leaving space for the at least one port, the at least five, at least ten or at least fifteen ports, respectively. Consequently, there is more freedom for choosing the shape and size of the at least one frontal ultrasonic sensor. Thus, it is easier to choose an optimal at least one frontal ultrasonic sensor for obtaining sonograms and thus ultrasound images and/or acoustic representations of interfaces, like for example the interface between brain tissue and the cerebrospinal fluid in the ventricle, located in front of the distal tip of the tubular body.

Alternatively, one or more of the at least one port, the at least five, at least ten or at least fifteen ports, respectively, is/are not arranged proximally from the at least one frontal ultrasonic sensor.

Preferably, the catheter further includes at least one lateral ultrasonic sensor, in particular at least one lateral ultrasonic transducer, for emitting lateral ultrasound waves and detecting the lateral ultrasound waves reflected back to the at least one lateral ultrasonic sensor, in particular for obtaining a sonogram, wherein the at least one lateral ultrasonic sensor is arranged laterally in the distal region of the tubular body. Thereby, in one example, the at least one lateral ultrasonic sensor is arranged laterally on the tubular body in the distal region of the tubular body and is thus arranged outside of the tubular body on the tubular body. In another example, the at least one lateral ultrasonic sensor is arranged laterally inside the tubular body in the distal region of the tubular body. For example, the at least one lateral ultrasonic sensor is arranged laterally in a sidewall of the tubular body in the distal region of the tubular body. In yet another example, the at least one lateral ultrasonic sensor is arranged behind the sidewall of the tubular body in the distal region of the tubular body. In either one of the two latter examples, the at least one lateral ultrasonic sensor is advantageously mounted on a support structure.

Independent of how the at least one lateral ultrasonic sensor is arranged laterally in the distal region of the tubular body, the at least one lateral ultrasonic sensor for emitting lateral ultrasound waves and detecting the lateral ultrasound waves reflected back to the at least one lateral ultrasonic sensor, in particular for obtaining a sonogram, has the advantage that sonograms and thus ultrasound images of the region around the distal region of the tubular body of the catheter can be obtained and/or that an acoustic signal representing an image and thus an acoustic representation of the region around the distal region of the tubular body of the catheter can be obtained. Thus, the navigation of the catheter can be improved during moving the catheter through the brain to the ventricle. Additionally, the visualization of the ventricles with the aid of the at least one lateral ultrasonic sensor provides important information of the ventricles'size. This information aids to adapt fast the drainage amount that is often based only on the intracranial pressure or information from external visualization such as head Computer Tomogram (CT) or Magnetic Resonance Imaging (MRI). Moreover, this information helps to distinguish the cause of a not draining catheter (result of empty drained ventricles, a clogged catheter or a catheter dislocation) because an empty drained ventricle and a dislocation of the catheter can be visualized. Furthermore, the monitoring of the proper positioning of the catheter in the ventricle during drainage of cerebrospinal fluid once the catheter is positioned in the ventricle can also be improved. Thus, an even more reliable and safe use of the catheter for drainage of fluid, in particular of cerebrospinal fluid is enabled.

Advantageously, the at least one lateral ultrasonic sensor is aligned to emit the lateral ultrasound waves away from the tubular body essentially in a direction in which the distal tip of the tubular body points. Thereby, the distal tip of the tubular body points along the direction of the longitudinal axis of the distal region of the tubular body and, when starting at the distal tip of the tubular body, faces away from the tubular body. Thus, emitting the lateral ultrasound waves away from the tubular body essentially in the direction in which the distal tip of the tubular body points preferably means that the lateral ultrasound waves are emitted in a direction inclined at an angle of less than 90°, particular preferably less than 50°, to the direction in which the distal tip of the tubular body points. Thereby, the lateral ultrasound waves may even be emitted in a direction parallel to the direction in which the distal tip of the tubular body points and thus along the tubular body towards the distal tip of the tubular body and beyond the distal tip of the tubular body.

The at least one lateral ultrasonic sensor being aligned to emit the lateral ultrasound waves away from the tubular body essentially in the direction in which the distal tip of the tubular body points has the advantage that sonograms and thus ultrasound images and/or acoustical representations of an area contacting the distal region of the tubular body of the catheter can be obtained. Thus, the navigation of the catheter can be further improved during moving the catheter through the brain to the ventricle. Furthermore, the monitoring of the proper positioning of the catheter in the ventricle during drainage of cerebrospinal fluid once the catheter is positioned in the ventricle is improved. Thus, an even more reliable and safer use of the catheter for drainage of liquid, in particular of cerebrospinal fluid, is enabled. These advantages are particularly pronounced in case each one of the at least one lateral ultrasonic sensor is aligned to emit the lateral ultrasound waves away from the tubular body essentially in the direction in which the distal tip of the tubular body points.

Alternatively, however, the at least one lateral ultrasonic sensor is not aligned to emit the lateral ultrasound waves away from the tubular body essentially in the direction in which the distal tip of the tubular body points.

Advantageously, the at least one lateral ultrasonic sensor is a piezoelectric ultrasonic transducer. Thus, advantageously, each one of the at least one lateral ultrasonic sensor includes a piezoelectric element, in particular a piezoelectric layer.

This has the advantage that the at least one lateral ultrasonic sensor can be designed particularly small and thus suitable for being arranged laterally in the distal region of the tubular body. In a variant, the at least one lateral ultrasonic sensor is a capacitive ultrasonic transducer. In yet another example, the at least one lateral ultrasonic sensor is neither a piezoelectric ultrasonic transducer nor a capacitive ultrasonic transducer. The at least one lateral ultrasonic sensor can also be another sensor than a transducer. For example, the at least one lateral ultrasonic sensor can include a transmitter for emitting the frontal ultrasound waves and a receiver for detecting the frontal ultrasound waves reflected back to the receiver and thus the lateral ultrasonic sensor, in particular for obtaining a sonogram, wherein the receiver is a unit separate from the transmitter. Thereby, the unit of the receiver and the unit of the transmitter can be arranged in one and same of the at least one lateral ultrasonic sensor.

Preferably, the at least one lateral ultrasonic sensor is adapted to emit the lateral ultrasound waves at a frequency in a range from 1 MHz to 20 MHz, particular preferably from 5 MHz the 12 MHz. This has the advantage that interfaces between brain tissue and cerebrospinal fluid can be detected and visualised in an ultrasound image up to a distance in a range from 4 cm to 10 cm from the respective one of the at least one lateral ultrasonic sensor. This has the advantage that the navigation of the catheter during moving the catheter through the brain to the ventricle is facilitated.

Preferably, the at least one lateral ultrasonic sensor is adapted to emit the lateral ultrasound waves with a peak rarefactional pressure in a range from 0 MPa to 7 MPa. This has the advantage that brain injuries during the use of the catheter due to the pressure caused by the frontal ultrasound waves can be avoided. Alternatively, however, the at least one lateral ultrasonic sensor is adapted to emit the lateral ultrasound waves with a peak rarefactional pressure of more than 7 MPa.

Advantageously, the at least one lateral ultrasonic sensor is adapted to emit the lateral ultrasound waves in a pulsed manner with 1 to 100 cycles in one pulse. Thereby, advantageously, the at least one lateral ultrasonic sensor is adapted to emit the lateral ultrasound waves at a pulse repetition frequency in a range from 100 Hz to 20 KHz. However, more than 100 cycles per pulse may be employed, too, and the pulse repetition frequency may be chosen to be smaller than 100 kHz or larger than 20 kHz.

Advantageously, the at least one frontal ultrasonic sensor and the at least one lateral ultrasonic sensor are adapted to provide in total an acoustic output level I_{SPTA.3} of 94 mW/cm² or less, advantageously I_{SPPA.3} of 190 W/cm² or less, wherein I_{SPTA.3} is the global maximum derated I_{SPTA} intensity value and I_{SPPA.3} is the Mechanical Index (or derated I_{SPPA} intensity) value indicated in Table 3 of “Marketing Clearance of Diagnostic Ultrasound Systems and Transducers”, “Guidance for Industry and Food and Drug Administration Staff” issued on Jun. 27, 2019, of the U.S. Department of Health and Human Services, Food and Drug Administration, Center for Devices and Radiological Health. Alternatively, however, the at least one frontal ultrasonic sensor and the at least one lateral ultrasonic sensor are adapted to provide in total a higher acoustic output level.

Alternatively, the at least one lateral ultrasonic sensor is adapted to emit the lateral ultrasound waves at a frequency of less than 1 MHz or more than 20 MHz. Advantageously, however, the at least one lateral ultrasonic sensor is adapted to emit the lateral ultrasound waves at a frequency of more than 18 kHz and less than 100 MHz, in particular less than 50 MHz.

Advantageously, the catheter includes at least 3 lateral ultrasonic sensors, preferably at least 15 lateral ultrasonic sensors, particular preferably at least 35 lateral ultrasonic sensors, most preferably at least 60 lateral ultrasonic sensors, for emitting lateral ultrasound waves and detecting the lateral ultrasound waves reflected back to the at least 3 lateral ultrasonic sensors, the at least 15 lateral ultrasonic sensors, the at least 35 lateral ultrasonic sensors or the at least 60 lateral ultrasonic sensors, respectively, in particular detecting the lateral ultrasound waves reflected back to respective one of the at least 3 lateral ultrasonic sensors, the at least 15 lateral ultrasonic sensors, the at least 35 lateral ultrasonic sensors or the at least 60 lateral ultrasonic sensors, respectively, in particular for obtaining a sonogram, wherein the at least 3 lateral ultrasonic sensors, the at least 15 lateral ultrasonic sensors, the at least 35 lateral ultrasonic sensors or the at least 60 lateral ultrasonic sensors, respectively, are laterally arranged in the distal region of the tubular body. This has the advantage that a more complete ultrasound image of the region around the distal region of the tubular body of the catheter can be obtained. Thus, the navigation of the catheter is further improved during moving the catheter through the brain to the ventricle. Furthermore, the monitoring of the proper positioning of the catheter in the ventricle during drainage of cerebrospinal fluid once the catheter is positioned in the ventricle is also further improved. Additionally, a size of the ventricle can be measured during moving the catheter through the brain to the ventricle and measured as well as monitored once the catheter is positioned in the ventricle. Thus, an even more reliable and safe use of the catheter for drainage of fluid, in particular of cerebrospinal fluid is enabled.

Advantageously, the catheter includes less than five hundred lateral ultrasonic sensors for emitting lateral ultrasound waves and detecting the lateral ultrasound waves reflected back to the less than five hundred lateral ultrasonic sensors, in particular detecting the lateral ultrasound waves reflected back to respective one of the less than five hundred lateral ultrasonic sensors, in particular for obtaining a sonogram, wherein the less than five hundred lateral ultrasonic sensors are laterally arranged in the distal region of the tubular body. Alternatively, the catheter includes five hundred or more lateral ultrasonic sensors for emitting lateral ultrasound waves and detecting the lateral ultrasound waves reflected back to the five hundred or more lateral ultrasonic sensors, in particular detecting the lateral ultrasound waves reflected back to respective one of the five hundred or more lateral ultrasonic sensors, in particular for obtaining a sonongram, wherein the five hundred or more lateral ultrasonic sensors are laterally arranged in the distal region of the tubular body.

In a preferred variant, the at least 3 lateral ultrasonic sensors, the at least 15 lateral ultrasonic sensors, the at least 35 lateral ultrasonic sensors or the at least 60 lateral ultrasonic sensors, respectively, are arranged in a lateral ultrasonic sensor region within in the distal region of the tubular body. Particular preferably, the lateral ultrasonic sensor region extends over a length in a range from about 0.5 cm to about 3 cm, in particular a range from about 1 cm to about 2 cm, along the tubular body. In a variant, however, the lateral ultrasonic sensor region extends over a length of less than about 0.5 cm or over a length of more than about 3 cm along the tubular body. Advantageously, each one of the at least 3 lateral ultrasonic sensors, the at least 15 lateral ultrasonic sensors, the at least 35 lateral ultrasonic sensors or the at least 60 lateral ultrasonic sensors, respectively, is a lateral ultrasonic transducer. This has the advantage that each one of the at least 3 lateral ultrasonic sensors, the at least 15 lateral ultrasonic sensors, the at least 35 lateral ultrasonic sensors or the at least 60 lateral ultrasonic sensors, respectively, can be designed particularly small and thus suitable for being arranged laterally in the distal region of the tubular body.

Alternatively, not all or even none of the at least 3 lateral ultrasonic sensors, the at least 15 lateral ultrasonic sensors, the at least 35 lateral ultrasonic sensors or the at least 60 lateral ultrasonic sensors, respectively, is a lateral ultrasonic transducer. For example, one, more than one or even all of the at least 3 lateral ultrasonic sensors, the at least 15 lateral ultrasonic sensors, the at least 35 lateral ultrasonic sensors or the at least 60 lateral ultrasonic sensors, respectively, can include a transmitter for emitting the frontal ultrasound waves and a receiver for detecting the frontal ultrasound waves reflected back to the receiver and thus the lateral ultrasonic sensor, in particular for obtaining a sonogram, wherein the receiver is a unit separate from the transmitter. Thereby, the unit of the receiver and the unit of the transmitter can be arranged in one and same of the at least 3 lateral ultrasonic sensors, the at least 15 lateral ultrasonic sensors, the at least 35 lateral ultrasonic sensors or the at least 60 lateral ultrasonic sensors, respectively.

Preferably, the at least 3 lateral ultrasonic sensors, the at least 15 lateral ultrasonic sensors, the at least 35 lateral ultrasonic sensors or the at least 60 lateral ultrasonic sensors, respectively, are distributed around a circumference of the tubular body to emit the lateral ultrasound waves in all directions, in particular in all 360 degrees around the circumference of the tubular body away from the tubular body. This has the advantage that a more complete ultrasound image of the region around the distal region of the tubular body of the catheter can be obtained. Thus, the navigation of the catheter is further improved during moving the catheter through the brain to the ventricle. Furthermore, the monitoring of the proper positioning of the catheter in the ventricle during drainage of cerebrospinal fluid once the catheter is positioned in the ventricle is also further improved. Thus, an even more reliable and safe use of the catheter for drainage of fluid, in particular of cerebrospinal fluid is enabled. Thereby, advantageously, the at least 3 lateral ultrasonic sensors, the at least 15 lateral ultrasonic sensors, the at least 35 lateral ultrasonic sensors or the at least 60 lateral ultrasonic sensors, respectively, are aligned to emit the lateral ultrasound waves away from the tubular body essentially in a direction in which the distal tip of the tubular body points. This has the advantage that a cone of lateral ultrasound waves opening in the direction of the distal tip of the tubular body is emitted by the at least 3 lateral ultrasonic sensors, the at least 15 lateral ultrasonic sensors, the at least 35 lateral ultrasonic sensors or the at least 60 lateral ultrasonic sensors, respectively, can be generated. This has the advantage that a field of ultrasound waves can be generated to obtain a panoramic view of the ventricular system around the tip of the tubular body, enabling estimating the size of the ventricle system.

In a particular preferred variant, at least 3 of the at least 3 lateral ultrasonic sensors, the at least 15 lateral ultrasonic sensors, the at least 35 lateral ultrasonic sensors or the at least 60 lateral ultrasonic sensors, respectively, provide a tubular shape, covering the circumference of the tubular body to emit the lateral ultrasound waves in all directions, in particular in all 360 degrees around the circumference of the tubular body away from the tubular body. With this variant, the before mentioned advantages for lateral ultrasonic sensors being the distributed around the circumference of the tubular body to emit the lateral ultrasound waves in all directions, in particular in all 360 degrees around the circumference of the tubular body away from the tubular body can be achieved in a particular easy and simple way.

Alternatively, however, the at least 3 lateral ultrasonic sensors, the at least 15 lateral ultrasonic sensors, the at least 35 lateral ultrasonic sensors or the at least 60 lateral ultrasonic sensors, respectively, are arranged differently in the distal region of the tubular body.

In case any of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, is a piezoelectric transducer, the respective one of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, advantageously includes a piezoelectric element, in particular a piezoelectric layer. Examples of piezoelectric ultrasonic transducers including such a piezoelectric element are described in the publication “Piezoelectric single crystal for ultrasonic transducers in biomedical application” of Qifa Zhou et al. in the Journal Progress in Materials Science, Volume 66, October 2014, Pages 87 to 111.

In case any of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, is a piezoelectric transducer, the respective one of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, advantageously includes an acoustic impedance matching layer or multiple acoustic impedance matching layers. The purpose of the one or multiple acoustic matching layers is to minimize the transmission loss due to acoustic impedance mismatch between the surface of the piezoelectric ultrasonic transducer and the tissue and/or liquid in which the piezoelectric ultrasonic transducer is operated. Advantageously, the piezoelectric element is coated with the one or multiple acoustic impedance matching layers. Thereby, the one or multiple acoustic impedance matching layers is advantageously coated on a front side of the piezoelectric element from which front side the ultrasound waves are to be emitted. The one or multiple acoustic impedance matching layers can for example be made from a polymer like an epoxy resin, polyurethane, polystyrene or parylene and may include further filler materials like for example silver particles. Thereby, the silver particles may for example have an average diameter of 2 μm to 3 μm.

The one or multiple acoustic impedance matching layers have the advantage that they provide a better energy transfer and thus enable a more efficient emittance of ultrasound waves. Thereby, parylene, which is a polymer whose backbone consists of para-benzenediyl rings —C₆H₄— connected by 1,2-ethanediyl bridges —CH₂—CH₂—, additionally has the advantage of acting at the same time as a protecting layer for the respective piezoelectric ultrasonic transducer.

Independent of whether the respective piezoelectric ultrasonic transducer includes such an acoustic impedance matching layer or multiple acoustic impedance matching layers or not, the respective piezoelectric ultrasonic transducer advantageously includes an acoustically absorbing backing layer. This has the advantage that the ultrasound waves emitted from the backside of the piezoelectric element can be absorbed by the acoustically absorbing backing layer. This enables preventing undesired effects resulting from the emission of ultrasound waves from the backside of the respective piezoelectric ultrasonic transducer which would reduce the ultrasound image quality obtainable with the respective piezoelectric ultrasonic transducer. Advantageously, the acoustically absorbing backing layer is coated on a backside of the piezoelectric element. In one example, the acoustically absorbing backing layer is made from sticky epoxy resin containing tungsten particles and silver particles. Such acoustically absorbing backing layers and methods for applying them on the piezoelectric element are for example described in U.S. Pat. No. 6,124,664 of Scimet Life Systems Inc. In another example, the acoustically absorbing backing layer comprises a composite of tungsten powder, cerium oxide powder in an amount from 1.0 weight percent to 4.5 weight percent tungsten, and an epoxy in a weight proportion to powder of from 4:1 to 50:1. Examples of such acoustically absorbing backing layers are described in U.S. Pat. No. 4,800,316 of Shanghai Lamp Factory.

The at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, can however as well be constructed differently.

Advantageously, the at least one frontal ultrasonic sensor is operatable in A-mode. In case the catheter includes more than one frontal ultrasonic sensors, the frontal ultrasonic sensors are advantageously operatable in A-mode or in B-Mode. In a variant, the at least one frontal ultrasonic sensor is operatable in another mode like for example in a Doppler mode or in harmonic mode.

Advantageously, the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, are operatable in A-mode or in B-mode. In a variant, the possible at least one lateral ultrasonic sensor, the possible at least 3 lateral ultrasonic sensors, the possible at least 15 lateral ultrasonic sensors, the possible at least 35 lateral ultrasonic sensors and the possible at least 60 lateral ultrasonic sensors, respectively, are operatable in another mode like for example in a Doppler mode or in harmonic mode.

A-mode is also referred to as amplitude mode and is for scanning a line through the body with the echoes plotted on screen as a function of depth. B-mode is also referred to as brightness mode where a linear array of transducers simultaneously scans a plane through the body that can be viewed as a two-dimensional image on screen. This mode is as well known as 2D mode. In harmonic mode, a transducer emits fundamental ultrasound pulses into the body and a central, narrow beam of harmonic overtones is reflected back after passing through the bodily tissues. When the harmonic mode is turned on, only this narrow beam of pulses is detected. The fundamental ultrasound pulses and scattered pulses are removed. Thus, lateral and contrast resolutions are improved with this mode.

Preferably, the at least one port, the at least five ports, at least ten ports or at least fifteen ports, respectively, is/are arranged distally from the at least one lateral ultrasonic sensor, the at least 3 lateral ultrasonic sensors, the at least 15 lateral ultrasonic sensors, at least 35 lateral ultrasonic sensors or at least 60 lateral ultrasonic sensors, respectively. This has the advantage that an efficient drainage of the liquid, in particular of the cerebrospinal fluid, from the ventricle into the drainage lumen is enabled without having to introduce the tip of the catheter too far into the ventricle, while at the same time, optimal ultrasound images from the area around the distal region with the at least one lateral ultrasonic sensor, the at least 3 lateral ultrasonic sensors, the at least 15 lateral ultrasonic sensors, at least 35 lateral ultrasonic sensors or at least 60 lateral ultrasonic sensors, respectively, can be obtained.

This advantage is particularly pronounced in case each one of the at least one port, the at least five ports, the at least ten ports or the at least fifteen ports, respectively, is/are arranged distally from the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively.

In case, the at least five ports, the at least ten ports, or the at least fifteen ports, respectively, are arranged in the above described port region within in the distal region of the tubular body, the port region is advantageously arranged distally from the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively. In case there are at least 3 lateral ultrasonic sensors, at least 15 lateral ultrasonic sensors, at least 35 lateral ultrasonic sensors or at least 60 lateral ultrasonic sensors, respectively, wherein the at least 3 lateral ultrasonic sensors, the at least 15 lateral ultrasonic sensors, the at least 35 lateral ultrasonic sensors or the at least 60 lateral ultrasonic sensors, respectively, are arranged in the above described lateral ultrasonic sensor region, the port region is advantageously arranged distally from the lateral ultrasonic sensor region. Thereby, the port region and the lateral ultrasonic sensor region are advantageously separate from each other and thus free of any overlap. In a variant however, the port region and the lateral ultrasonic sensor region are at least partially overlapping.

In a variant, however, the at least one port, the at least five ports, at least ten ports or at least fifteen ports, respectively, are not arranged distally from the at least one lateral ultrasonic sensor, the at least 3 lateral ultrasonic sensors, the at least 15 lateral ultrasonic sensors, at least 35 lateral ultrasonic sensors or at least 60 lateral ultrasonic sensors, respectively.

As an alternative to all these variants with the at least one lateral ultrasonic sensor, the catheter goes without the at least one lateral ultrasonic sensor.

Advantageously, the tubular body is made from a flexible material. This flexible material can for example be a synthetic material like polyimide or polyurethane. This has the advantage that fewer injuries are inflicted to the brain after insertion of the catheter to the ventricle.

Alternatively, however, the tubular body is not made from a flexible material but is made from a stiff material.

Independent of whether the tubular body is made from a flexible material or not, the tubular body is advantageously coated with an antimicrobial coating. This has the advantage that the risk of infections caused by the use of the catheter is reduced. For example, a combination of clindamycin rifampin, a combination of minocycline and rifampin or a coating containing silver coating can be used as antimicrobial coating. Examples of such coatings are described in the publication “Clinical review: Efficacy of antimicrobial-impregnated catheters in external ventricular drainage—a systematic review and meta-analysis” of Xiang Wang et al., Critical Care 17, 234 (2013).

Alternatively, however, the tubular body goes without being coated with an antimicrobial coating.

Advantageously, the catheter has a length in a range from 20 cm to 30 cm. This has the advantage that the catheter is long enough for being able to reach to the ventricle while not being too long to become bulky.

Alternatively, however, the catheter can have a length of less than 20 cm or more than 30 cm.

Preferably, the tubular body has an outer diameter in a range from 2 mm to 10 mm, particular preferably in a range from 2 mm to 4 mm. This has the advantage that the internal drainage lumen running along the tubular body for the drainage of the liquid can be designed to have a large enough diameter for an effective drainage of the liquid while at the same time, the catheter maintains an outer diameter small enough to not inflict unnecessary injuries to the brain.

Alternatively, however, the tubular body may have an outer diameter of less than 2 mm or of more than 10 mm.

Preferably, the drainage lumen provides an inner diameter in a range from 1 mm to 8 mm, particular advantageously 1 mm to 2 mm. This has the advantage that the internal drainage lumen running along the tubular body for the drainage of the liquid is large enough for an effective drainage of the liquid. Thereby, the inner diameter of drainage lumen is advantageously smaller than the outer diameter of the tubular body. Particular advantageously, the inner diameter of the drainage lumen is at least 0.5 mm smaller, more advantageously at least 1.0 mm smaller, than the outer diameter of the tubular body. This has the advantage that the tubular body can be constructed to have a sufficient stability. Nonetheless, the outer diameter of the tubular body is preferably smaller than the inner diameter of the drainage lumen plus 5.0 mm.

Advantageously, the catheter includes an intracranial pressure sensor for measuring an intracranial pressure when the catheter is inserted to the ventricle. This has the advantage that the intracranial pressure can be determined during use of the catheter.

In an advantageous variant, one of the at least one frontal ultrasonic sensor or one of the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, is at the same time an intracranial pressure sensor. This has the advantage that the catheter can be constructed more compact. In another advantageous variant, however, the intracranial pressure sensor is a sensor which is separate from the at least one frontal ultrasonic sensor or one of the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively. This has the advantage that the intracranial pressure can be determined independent of the at least one frontal ultrasonic sensor or one of the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively.

In an alternative to these variants, the catheter goes without an intracranial pressure sensor for measuring an intracranial pressure when the catheter is inserted to the ventricle.

Advantageously, the catheter includes a wiring connecting the at least one frontal ultrasonic sensor with a connector for being connected to a control unit for controlling the at least one frontal ultrasonic sensor, the connector being arranged in a proximal region of the catheter. This has the advantage that the catheter can be separated from the control unit for maintenance purposes and connected to the control unit for controlling the at least one frontal ultrasonic sensor when using the catheter for placement in a ventricular system, in particular of a human, for drainage of liquid, in particular of cerebrospinal fluid, from a ventricle of the ventricular system.

Alternatively, however, the catheter goes without such a connector. In such a case, the catheter advantageously includes a wiring connecting the at least one frontal ultrasonic sensor with the control unit for controlling the at least one frontal ultrasonic sensor, the connector being arranged in a proximal region of the catheter.

In case the catheter further includes the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, for emitting lateral ultrasound waves and detecting the lateral ultrasound waves reflected back to the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, in particular for obtaining a sonogram, wherein the at least one lateral ultrasonic sensor is arranged laterally in the distal region of the tubular body, the catheter advantageously includes a wiring connecting the at least one lateral ultrasonic sensor, the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, with a connector for being connected to a control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, the connector being arranged in the proximal region of the catheter. This has the advantage that the catheter can be separated from the control unit for maintenance purposes and connected to the control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, when using the catheter for placement in a ventricular system, in particular of a human, for drainage of liquid, in particular of cerebrospinal fluid, from a ventricle of the ventricular system.

Thereby, in case the catheter also includes the above mentioned wiring connecting the at least one frontal ultrasonic sensor with a connector for being connected to a control unit for controlling the at least one frontal ultrasonic sensor, the connector being arranged in a proximal region of the catheter, advantageously, the connector for being connected to a control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, is at the same time the connector for being connected to a control unit for controlling the at least one frontal ultrasonic sensor. In a variant however, these are two separate connectors.

Furthermore, in case the catheter also includes the above mentioned wiring connecting the at least one frontal ultrasonic sensor with a connector for being connected to a control unit for controlling the at least one frontal ultrasonic sensor, the control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, is at the same time the control unit for controlling the at least one frontal ultrasonic sensor. In a variant however, these are two separate control units.

Alternatively to these variants, however, the catheter goes without such a connector. In such a case, the catheter advantageously includes a wiring connecting the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, with the control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, the connector being arranged in a proximal region of the catheter.

In a preferred variant, the catheter includes a wiring connecting the at least one frontal ultrasonic sensor and the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, with the connector for being connected or being connected to a control unit for controlling the at least one frontal ultrasonic sensor and the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, the connector being arranged in the proximal region of the catheter.

Independent of whether the control unit for controlling the at least one frontal ultrasonic sensor and the control unit for controlling the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, are one and the same control unit or separate control units, the control unit(s) are advantageously adapted for controlling the respective ultrasonic sensor or ultrasonic sensors, respectively, to emit the frontal ultrasound waves and/or the lateral ultrasound waves, respectively. Furthermore, the control unit(s) are advantageously adapted to receive a signal from the reflected frontal ultrasound waves received by the at least one frontal ultrasonic sensor, in particular for obtaining sonograms, or from the reflected lateral ultrasound waves received by the at least one lateral ultrasonic sensor, the at least 3 lateral ultrasonic sensors, the at least 15 lateral ultrasonic sensors, the at least 35 lateral ultrasonic sensors or the at least 60 lateral ultrasonic sensors, respectively, in particular for obtaining sonograms. Furthermore, the control unit(s) are advantageously adapted to calculate ultrasound images and thus sonograms based on the received signals and/or adapted to calculate an acoustic signal representing the sonogram based on the received signals. In a advantageous variant, however, the control unit(s) are connectable to a separate calculation unit adapted to calculate the ultrasound images and thus sonograms based on the received signals and/or adapted to calculate an acoustic signal representing the sonogram based on the received signals. Thereby, the separate calculation unit can be a personal computer (PC), a tablet, a smartphone or any other device including a processor which is adapted to calculate the ultrasound images and thus sonograms or acoustic signal being an acoustic representation of the sonogram based on the received signals.

Advantageously, the wiring of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, includes at least n+1 electrical connections, wherein n is the total number of sensors being the sum of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively.

Advantageously, the wiring of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, is a printed circuit board (PCB), in particular a high density interconnect printed circuit board (HDI PCB). Thereby, the printed circuit board advantageously includes a flexible substrate. The flexible substrate can for example be made from polyimide, polyester, polyethylene nphtalate, polytetrafluoroetghylene (PTFE) or aromathic polyamide. Particular advantageously, the flexible substrate is made from a polyimide film, a polyester film, a polyethylene nphtalate film, a polytetrafluoroetghylene (PTFE) film or an aromathic polyamide film. One example of a polyimide film is Kapton. Advantageously, the polyimide film has a thickness of less than 200 μm, in particular less than 100 μm.

The printed circuit board is advantageously terminated with the connector. Thereby, the connector can be a custom made connector or a standard connector to make the electrical connections to the control unit.

Alternatively, however, the wiring of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, is not a printed circuit board (PCB) but made from wires.

Advantageously, the catheter is magnetic resonance imaging compatible, in particular magnetic resonance imaging safe. Thus, the catheter is advantageously usable in a patient in a magnetic resonance imaging (MRI) apparatus. Thereby, the magnetic resonance imaging (MRI) apparatus is advantageously a magnetic resonance imaging (MRI) apparatus employing a magnetic field of up to 3 Tesla. In this magnetic field, the catheter does not heat up, does not move in the brain of the patient and thus does not cause injuries to the patient in the magnetic resonance imaging (MRI) apparatus. Furthermore, the catheter does not cause artefacts in the images obtained with the magnetic resonance imaging (MRI) apparatus. In an alternative however, the catheter is not magnetic resonance imaging compatible.

Independent of how the wiring is made, the control unit for controlling the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, and the catheter according to the invention are advantageously used and sold in combination. Thus, a combination of such a control unit with the catheter as described above is advantageous.

The control unit is advantageously adapted for controlling the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively. Thus, the control unit is advantageously adapted to apply excitation signals at a pre-set amplitude and having a phase information to each one of the at least one frontal ultrasonic sensor and the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, when being connected to the catheter. Furthermore, the control unit is advantageously adapted to receive signals from the at least one frontal ultrasonic sensor including information on the frontal ultrasound waves reflected back to the at least one frontal ultrasonic sensor and detected with the at least one frontal ultrasonic sensor. Furthermore, the control unit is advantageously adapted to receive signals from the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, the signals including information on the lateral ultrasound waves reflected back to the respective one of the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively, and detected with respective one of the possible at least one lateral ultrasonic sensor, the possible at least 3 lateral ultrasonic sensors, the possible at least 15 lateral ultrasonic sensors, the possible at least 35 lateral ultrasonic sensors or the possible at least 60 lateral ultrasonic sensors, respectively.

The control unit is advantageously adapted to operate the at least one frontal ultrasonic sensor in A-mode. In case the catheter includes more than one frontal ultrasonic sensors, the control unit is advantageously adapted to operate the frontal ultrasonic sensors in A-mode or in B-mode. Thereby, in an advantageous variant, the control unit is adapted to operate the at least one frontal ultrasonic sensor in B-mode to provide ultrasound images focussed on a plane being oriented perpendicular to the longitudinal axis of the distal region of the tubular body and being located at a distance from the distal tip of the tubular body. Thereby, the control unit is advantageously adapted to sweep the distance for obtaining ultrasound images at the different distances during sweeping the distance.

In another variant, the control unit is adapted to operate the at least one frontal ultrasonic sensor in another mode like for example in a Doppler mode or in harmonic mode.

The control unit is advantageously adapted to operate the possible at least 3 lateral ultrasonic sensors, the possible at least 15 lateral ultrasonic sensors, the possible at least 35 lateral ultrasonic sensors or the possible at least 60 lateral ultrasonic sensors, respectively, in A-mode or in B-mode. Thereby, in an advantageous variant, the control unit is adapted to operate the possible at least 3 lateral ultrasonic sensors, the possible at least 15 lateral ultrasonic sensors, the possible at least 35 lateral ultrasonic sensors or the possible at least 60 lateral ultrasonic sensors in B-mode to provide ultrasound images focussed on a plane being oriented perpendicular to the longitudinal axis of the distal region of the tubular body and being located at a distance from the distal tip of the tubular body. Thereby, the control unit is advantageously adapted to sweep the distance for obtaining ultrasound images at the different distances during sweeping the distance. In another variant, the control unit is adapted to operate the possible at least one lateral ultrasonic sensor, the possible at least 15 lateral ultrasonic sensors, the possible at least 35 lateral ultrasonic sensors and the possible at least 60 lateral ultrasonic sensors, respectively, in another mode like for example in a Doppler mode or in harmonic mode.

Furthermore, the control unit is advantageously adapted to calculate in real time ultrasound images and thus sonograms based on the received signals and/or adapted to calculate in real time an acoustic signal representing the sonogram based on the received signals.

In case the control unit is adapted to calculate the sonograms in real time, the control unit advantageously includes a display for displaying the calculated sonograms. In a variant, however, the control unit is directly or indirectly connectable to a separate display for displaying the calculated sonograms. In case the control unit is adapted to calculate in real time an acoustic signal representing the sonogram, the control unit advantageously includes an audio output for outputting the calculated acoustic signal. Thereby, the audio output can be a loudspeaker or headphones or an output for connecting directly or indirectly to a loudspeaker or headphones.

In an advantageous variant, however, the control unit is connectable to a separate calculation unit adapted to calculate in real time the ultrasound images and thus sonograms based on the received signals and/or adapted to calculate in real time the acoustic signal representing the sonogram based on the received signals. Thereby, the separate calculation unit can be a personal computer (PC), a tablet, a smartphone or any other device including a processor which is adapted to calculate the ultrasound images and thus sonograms or acoustic signal being an acoustic representation of the sonogram based on the received signals.

Alternatively, the control unit and the catheter can be sold individually.

Advantageously, the catheter according to the invention is used in an arrangement including a mandrin, in particular a stylet, and the catheter. Thereby, a mandrin is a guide for the catheter. Advantageously, a mandrin is a stiff wire or stylet inserted into the catheter and gives the catheter a shape and firmness while passing through the brain to the ventricle. Thus, advantageously, an arrangement includes a mandrin, in particular a stylet and the catheter according to the invention. In an advantageous variant, the arrangement further includes the above described control unit. Thus, the arrangement advantageously includes the mandrin, in particular the stylet and the above described the combination of the control unit and the catheter.

Alternatively, however, the combination of the control unit and the catheter can be used without the mandrin. Even more, the catheter can be used and sold independent of the madrin and independent of the control unit.

Other advantageous embodiments and combinations of features come out from the detailed description below and the entirety of the claims.

### BRIEF DESCRIPTION OF THE DRAWINGS

The drawings used to explain the embodiments show:

FIG. 1 a simplified schematic view of an arrangement including a stylet and a combination of a control unit and a catheter according to the invention,

FIG. 2 a simplified schematic view of cross section through the catheter shown in FIG. 1, wherein the cross section is positioned along a tubular body of the catheter at a position within a lateral ultrasonic sensor region within a distal region of the tubular body, and

FIG. 3 a simplified schematic view of cross section through another catheter according to the invention, wherein the cross section is positioned along the tubular body of the catheter at a position within the lateral ultrasonic sensor region within the distal region of the tubular body.

In the figures, the same components are given the same reference symbols.

### PREFERRED EMBODIMENTS

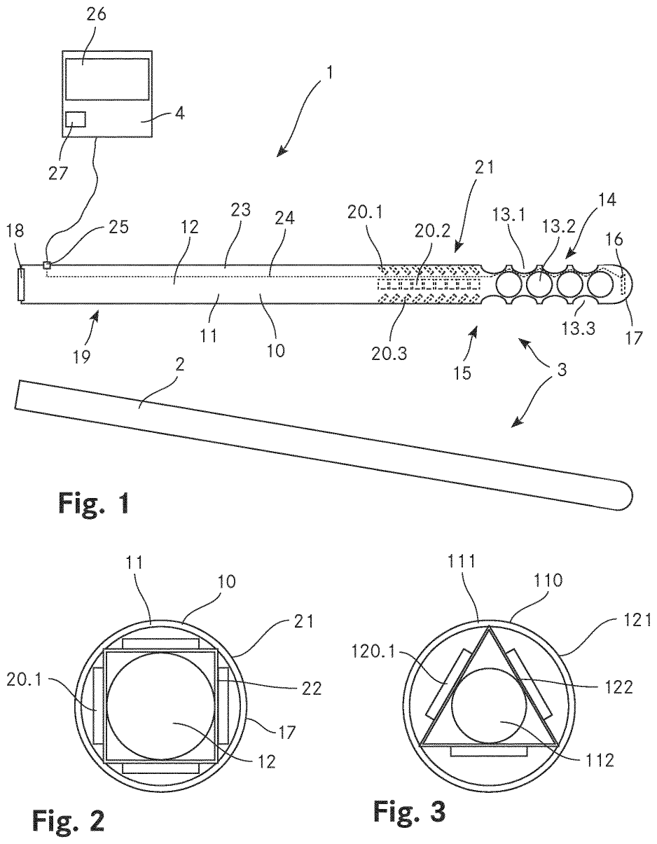

FIG. 1 shows a simplified schematic view of an arrangement 1 including a stylet 2 and a combination 3 of a control unit 4 and a catheter 10 according to the invention. Thus, the arrangement 1 includes the stylet 2 and the catheter 10.

The catheter 10 is for placement in a ventricular system, in particular of a human, for drainage of liquid, in particular of cerebrospinal fluid (CSF), from a ventricle of the ventricular system. More precisely, the catheter 10 is an external ventricular drainage catheter. This catheter 10 includes a tubular body 11 having an internal drainage lumen 12 running along the tubular body 11 for the drainage of the liquid. Thereby, the tubular body 11 is made from a flexible material. More precisely, the tubular body 11 is made from polyimide. In a variant, the tubular body 11 is made from a different material like for example polyurethane. In yet another variant, the tubular body 11 is made from a stiff material. Independent of the material the tubular body is made from, in the embodiment shown in FIG. 1, the tubular body 11 is made from a tube having an internal lumen forming the drainage lumen 12. Thereby, the tubular body 11 is coated with an antimicrobial coating 23. In a variant, the catheter 10 goes without an antimicrobial coating of the tubular body.

The tubular body 11 includes sixteen ports 13.1, 13.2, 13.3 arranged in a port region 14 in a distal region 15 of the tubular body 11. These sixteen ports 13.1, 13.2, 13.3 are arranged laterally in a sidewall of the tubular body 11. Each one of the sixteen ports 13.1, 13.2, 13.3 connects the drainage lumen 12 with an outside of the tubular body 11 and outside of the catheter 10 for the drainage of the liquid from the ventricle into the drainage lumen 12. The sixteen ports 13.1, 13.2, 13.3 are distributed around a circumference of the tubular body 11 to face in all directions around a circumference of the tubular body 11. Thus in all 360 degrees around the circumference of the tubular body 11, an opening in the sidewall of the tubular body 11 is provided by at least one of the sixteen ports 13.1, 13.2, 13.3.

The port region 14 extends over a length in a range from about 1 cm to about 2 cm along the tubular body 11. In a variant, the port region 14 extends over a length in a range from about 1.5 cm to about 2.0 cm along the tubular body 11.

In variants, the catheter may as well include more than sixteen ports or less than sixteen ports. In one example, the catheter includes only one port. In another example, the catheter includes four ports. In yet another example, the catheter includes six ports. In yet another example, the catheter includes ten ports.

As shown in FIG. 1, the tubular body 11 includes an outlet 18 for letting the liquid from the drainage lumen 12 out of the tubular body 11, the outlet 18 being arranged in a proximal region 19 of the tubular body 11. Thus, a liquid collection volume which is not shown here can be connected to the outlet 18 for the drainage of the liquid from the drainage lumen 12.

The catheter 10 further includes a frontal ultrasonic sensor 16 for emitting frontal ultrasound waves and detecting the frontal ultrasound waves reflected back to the frontal ultrasonic sensor 16. This frontal ultrasonic sensor 16 is aligned to emit the frontal ultrasound waves from the distal tip 17 of the tubular body 11 away from the tubular body 11 in a direction along a longitudinal axis of the distal region 15 of the tubular body 11. This frontal ultrasonic sensor 16 is a frontal ultrasonic transducer and is arranged at a distal tip 17 of the tubular body 11 being at the same time the distal tip of the catheter 10. More precisely, the frontal ultrasonic transducer is a piezoelectric ultrasound transducer and is adapted to emit the frontal ultrasound waves at a frequency in a range from 5 MHz to 12 MHz. In a variant, the frontal ultrasonic sensor 16 is adapted to emit the frontal ultrasound waves at another frequency like for example 20 kHz, 15 MHz, 18 MHz or 50 MHz. Independent of the frequency, the frontal ultrasonic sensor 16 can as well be another type of sensor like for example a capacitive ultrasonic transducer.

In a variant, however, the catheter includes more than one frontal ultrasonic sensors for emitting frontal ultrasound waves and detecting the frontal ultrasound waves reflected back to the frontal ultrasonic sensors. In one example, the catheter includes three frontal ultrasonic sensors for emitting frontal ultrasound waves and detecting the frontal ultrasound waves reflected back to the three frontal ultrasonic sensors. These three frontal ultrasonic sensors are aligned to emit the frontal ultrasound waves from the distal tip of the tubular body away from the tubular body in a direction inclined at an angle of 5° to the longitudinal axis of the distal region of the tubular body and which are inclined to each other by at least 5°.

The frontal ultrasonic sensor or the frontal ultrasonic sensors, respectively, are adapted to emit the frontal ultrasound waves with a peak rarefactional pressure of 1 MPa. In a variant, the frontal ultrasonic sensor or the frontal ultrasonic sensors, respectively, are adapted to emit the frontal ultrasound waves with a peak rarefactional pressure of 3 MPa. In yet another variant, the frontal ultrasonic sensor or the frontal ultrasonic sensors, respectively, are adapted to emit the frontal ultrasound waves with a peak rarefactional pressure of 6 MPa.

Furthermore, the frontal ultrasonic sensor or the frontal ultrasonic sensors, respectively, are adapted to adapted to emit the frontal ultrasound waves in a pulsed manner with 1 cycle in one pulse. Thereby, the frontal ultrasonic sensor or the frontal ultrasonic sensors, respectively, are adapted to emit the frontal ultrasound waves at a pulse repetition frequency of 100 Hz. In a variant, the frontal ultrasonic sensor or the frontal ultrasonic sensors, respectively, are adapted to emit the frontal ultrasound waves at a pulse repetition frequency of 1 kHz. In another variant, the frontal ultrasonic sensor or the frontal ultrasonic sensors, respectively, are adapted to emit the frontal ultrasound waves at a pulse repetition frequency of 10 kHz. In yet another variant, the frontal ultrasonic sensor or the frontal ultrasonic sensors, respectively, are adapted to emit the frontal ultrasound waves at a pulse repetition frequency of 20 kHz.

From the signals of the emitted frontal ultrasound waves and the detected frontal ultrasound waves reflected back to the frontal ultrasonic sensor 16, ultrasound images of interfaces, like for example the interface between brain tissue and the cerebrospinal fluid in the ventricle, located in front of the distal tip 17 of the tubular body 11 can be calculated by the control unit 4. These ultrasound images are sonograms. Furthermore, in addition of obtaining these sonograms, from the signals of the emitted frontal ultrasound waves and the detected frontal ultrasound waves reflected back to the frontal ultrasonic sensor 16, an acoustic signal representing an image of the interfaces, like for example the interface between brain tissue and the cerebrospinal fluid in the ventricle, located in front of the distal tip 17 of the tubular body 11 can be calculated by the control unit 4.

Due to the frontal ultrasonic sensor 16 for emitting the frontal ultrasound waves and detecting the frontal ultrasound waves reflected back to the frontal ultrasonic sensor 16 a real time visualisation and acoustic representation of the interfaces in front of the catheter 10, like for example the interface between brain tissue and the cerebrospinal fluid in the ventricle in front of the catheter 10, can be obtained. Consequently, due to the frontal ultrasonic sensor 16, the navigation of the catheter 10 in the brain and the ventricle is facilitated.

The sixteen ports 13.1, 13.2, 13.3 are arranged proximally from the frontal ultrasonic sensor 16. Thus, the port region 14 is arranged proximally from the frontal ultrasonic sensor 16.

The catheter 10 further includes 36 lateral ultrasonic sensors 20.1, 20.2, 20.3 for emitting lateral ultrasound waves and detecting the lateral ultrasound waves reflected back to the lateral ultrasonic sensors 20.1, 20.2, 20.3. These lateral ultrasonic sensors 20.1, 20.2, 20.3 are lateral ultrasonic transducers, namely lateral piezoelectric ultrasonic transducers, and are arranged laterally in a lateral ultrasonic sensor region 21 in the distal region 15 of the tubular body 11. Thereby, the lateral ultrasonic sensor region 21 extends over a length in a range from about 0.5 cm to about 3 cm along the tubular body 11. In a variant, the lateral ultrasonic sensor region 21 extends over a length in a range from about 1 cm to about 2 cm along the tubular body. Independent of the length of the lateral ultrasonic sensor region 21, the lateral ultrasonic sensors 20.1, 20.2, 20.3 are arranged laterally in the sidewall of the tubular body 11 and are mounted on a support structure 22 as shown in the context of FIGS. 2 and 3.

The lateral ultrasonic sensors 20.1, 20.2, 20.3 are distributed around the circumference of the tubular body 11 to emit the lateral ultrasound waves in all directions, in particular in all 360 degrees around the circumference of the tubular body 11 away from the tubular body 11. In a variant, however, the lateral ultrasonic sensors 20.1, 20.2, 20.3 are located around less than 360 degrees of the circumference or even only on one sight of the catheter. Furthermore, the lateral ultrasonic sensors 20.1, 20.2, 20.3 are aligned to emit the lateral ultrasound waves away from the tubular body 11 in directions inclined at an angle of 45° to the direction in which the distal tip 17 of the tubular body 11 points. Thus, the lateral ultrasonic sensors 20.1, 20.2, 20.3 are aligned to emit the lateral ultrasound waves away from the tubular body 11 essentially in a direction in which the distal tip 17 of the tubular body 11 points. Thereby, the distal tip 17 of the tubular body 11 points along the direction of the longitudinal axis of the distal region 15 of the tubular body 11 and, when starting at the distal tip 17 of the tubular body 11, faces away from the tubular body 11. In variants, the lateral ultrasonic sensors 20.1, 20.2, 20.3 are aligned differently. In one example, the lateral ultrasonic sensors 20.1, 20.2, 20.3 are aligned to emit the lateral ultrasound waves away from the tubular body 11 in a direction inclined at an angle of 50° to the direction in which the distal tip 17 of the tubular body 11 points. In another example, the lateral ultrasonic sensors 20.1, 20.2, 20.3 are aligned to emit the lateral ultrasound waves away from the tubular body 11 in a direction inclined at an angle of 85° to the direction in which the distal tip 17 of the tubular body 11 points.

The lateral ultrasonic sensors 20.1, 20.2, 20.3 are adapted to emit the lateral ultrasound waves at a frequency in a range from 5 MHz to 12 MHz. In a variant, the lateral ultrasonic sensors 20.1, 20.2, 20.3 are adapted to emit the frontal ultrasound waves at another frequency like for example 20 kHz, 15 MHz, 18 MHz or 50 MHz. Independent of the frequency, the lateral ultrasonic sensors 20.1, 20.2, 20.3 can as well be another type of sensor like for example a capacitive ultrasonic transducer.

The lateral ultrasonic sensors 20.1, 20.2, 20.3 are adapted to emit the lateral ultrasound waves with a peak rarefactional pressure of 1 MPa. In another variant, the lateral ultrasonic sensors 20.1, 20.2, 20.3 are adapted to emit the lateral ultrasound waves with a peak rarefactional pressure of 3 MPa. In yet another variant, the lateral ultrasonic sensors 20.1, 20.2, 20.3 are adapted to emit the lateral ultrasound waves with a peak rarefactional pressure of 6 MPa. Furthermore the lateral ultrasonic sensors 20.1, 20.2, 20.3 are adapted to emit the lateral ultrasound waves in a pulsed manner with 1 cycle in one pulse. Thereby, the lateral ultrasonic sensors 20.1, 20.2, 20.3 are adapted to emit the lateral ultrasound waves at a pulse repetition frequency of 100 Hz. In a variant, the lateral ultrasonic sensors 20.1, 20.2, 20.3 are adapted to emit the lateral ultrasound waves at a pulse repetition frequency of 1 kHz. In yet another variant, the lateral ultrasonic sensors 20.1, 20.2, 20.3 are adapted to emit the lateral ultrasound waves at a pulse repetition frequency of 10 kHz. In yet another variant, the lateral ultrasonic sensors 20.1, 20.2, 20.3 are adapted to emit the lateral ultrasound waves at a pulse repetition frequency of 20 kHz. One of the lateral ultrasonic sensors 20.1 is at the same time an intracranial pressure sensor for measuring an intracranial pressure when the catheter is inserted to the ventricle. In a variant, however, the intracranial pressure sensor is a sensor separate from the frontal ultrasonic sensor and the lateral ultrasonic sensors. In yet another variant, the catheter goes without an intracranial pressure sensor for measuring an intracranial pressure when the catheter is inserted to the ventricle.

In a variant, the catheter includes only one lateral ultrasonic sensor. In another variant, the catheter includes 15 lateral ultrasonic sensors. In yet another variant, the catheter includes 60 lateral ultrasonic sensors. In yet another variant, the catheter includes 72 or even more lateral ultrasonic sensors.

Independent of the number of lateral ultrasonic sensors, the sixteen ports 13.1, 13.2, 13.3 are arranged distally from the 36 lateral ultrasonic sensors 20.1, 20.2, 20.3. Thus, the port region 14 is arranged distally from the lateral ultrasonic sensor region 21. Thereby, the port region 14 and the lateral ultrasonic sensor region 21 are separate from each other and thus free of any overlap.

The catheter 10 has a length of 30 cm. In a variant, the length of the catheter is however longer than 30 cm. In another variant, the catheter 10 has a length of 25 cm. In yet another variant, the catheter 10 has a length of 20 cm. In yet other variants, the catheter 10 has a length of less than 20 cm. The tubular body 11 has an outer diameter of 4 mm and the drainage lumen 12 has an inner diameter of 2 mm. The outer diameter of the tubular body 11 can however also be smaller or larger than 4 mm. As examples, the outer diameter of the tubular body 11 can be 2 mm, 5 mm, 6 mm, 8 mm or 10 mm. Similarly, the inner diameter of the drainage lumen 12 can be smaller or larger than 2 mm. As examples, the inner diameter of the drainage lumen 12 can be 1 mm, 4 mm, 5 mm, 6 mm or 8 mm. Advantageously, however the inner diameter of the drainage lumen 12 is at least 0.5 mm or even at least 1.0 mm smaller than the outer diameter of the tubular body 11.

As shown in FIG. 1, the catheter 10 includes a wiring 24 connecting the frontal ultrasonic sensor 16 and the lateral ultrasonic sensors 20.1, 20.2, 20.3 with a connector 25 for being connected to the control unit 4 for controlling the frontal ultrasonic sensor 16 and for controlling the lateral ultrasonic sensors 20.1, 20.2, 20.3. Thereby, the connector 25 is arranged in the proximal region 19 of the catheter 10.

The wiring 24 of the frontal ultrasonic sensor 16 and the 36 lateral ultrasonic sensors includes at 38 electrical connections. Thus, the wiring 24 includes n+1 electrical connections, wherein n is the total number of sensors being the sum of the frontal ultrasonic sensor 16 and the 36 lateral ultrasonic sensors. The wiring 24 is a high density interconnect printed circuit board (HDI PCB) including a flexible substrate made of Kapton having a thickness of 125 μm. In variants, however, the wiring 24 can be constructed differently. For example, the HDI PCB can include a different flexible substrate or a stiff substrate. In yet another variant, the wiring can be made of individual wires instead of from a printed circuit board.

The control unit 4 is adapted for controlling the frontal ultrasonic sensor 16 and the lateral ultrasonic sensors 20.1, 20.2, 20.3. Thereby, the control unit 4 is adapted to apply excitation signals at a pre-set amplitude and having a phase information to each one of the frontal ultrasonic sensor 16 and the lateral ultrasonic sensors 20.1, 20.2, 20.3, when being connected to the catheter 10. Furthermore, the control unit 4 is adapted to receive signals from the frontal ultrasonic sensor 16 including information on the frontal ultrasound waves reflected back to the frontal ultrasonic sensor 16 and detected with the frontal ultrasonic sensor 16. Thereby, the control unit 4 is adapted to operate the frontal ultrasonic sensor 16 in A-mode. In order to enable this, the frontal ultrasonic sensor 16 is operatable in A-mode. In the mentioned variant where the catheter includes more than one frontal ultrasonic sensor, the control unit is however adapted to operate the frontal ultrasonic sensors in A-mode or in B-mode. In order to enable this, the frontal ultrasonic sensors are operatable in A-mode or in B-mode. Thereby, the control unit 4 is adapted to operate the frontal ultrasonic sensors in B-mode to provide ultrasound images focussed on a plane being oriented perpendicular to the longitudinal axis of the distal region 15 of the tubular body 11 and being located at a distance from the distal tip 17 of the tubular body 11. Thereby, the control unit 4 is adapted to sweep the distance for obtaining ultrasound images at the different distances during sweeping the distance.

Furthermore, the control unit 4 is adapted to receive signals from the lateral ultrasonic sensors 20.1, 20.2, 20.3, the signals including information on the lateral ultrasound waves reflected back to the respective one of the lateral ultrasonic sensors 20.1, 20.2, 20.3, and detected with respective one of the lateral ultrasonic sensors 20.1, 20.2, 20.3. Thereby, the control unit 4 is adapted to operate the lateral ultrasonic sensors 20.1, 20.2, 20.3 in A-mode and to operate the lateral ultrasonic sensors 20.1, 20.2, 20.3 in B-mode. Thus, the lateral ultrasonic sensors 20.1, 20.2, 20.3 are operatable in A-mode and operatable in B-mode. Thereby, the control unit 4 is adapted to operate the lateral ultrasonic sensors 20.1, 20.2, 20.3 in B-mode to provide ultrasound images focussed on a plane being oriented perpendicular to the longitudinal axis of the distal region 15 of the tubular body 11 and being located at a distance from the distal tip 17 of the tubular body 11. Thereby, the control unit 4 is adapted to sweep the distance for obtaining ultrasound images at the different distances during sweeping the distance.

The control unit 4 is adapted to calculate in real time ultrasound images and thus sonograms based on the received signals and adapted to calculate in real time an acoustic signal representing the sonogram based on the signals received from the frontal ultrasonic sensor 16 and the lateral ultrasonic sensors 20.1, 20.2, 20.3. The control unit 4 further includes a display 26 for displaying the calculated sonograms and a loudspeaker 27 for outputting the acoustic signal.

FIG. 2 shows a simplified schematic view of cross section through the catheter 10 shown in FIG. 1. Thereby, the cross section is positioned along the tubular body 11 at a position within the lateral ultrasonic sensor region 21 within the distal region of the tubular body 11. Thus, FIG. 2 shows that the support structure 22 on which the ultrasonic sensors 20.1 are mounted has a quadratic cross section and surrounds the drainage lumen 12 having a circular cross section and running along the tubular body 11. Thereby, the ultrasonic sensors 20.1 are mounted on the outer surfaces of the four sides of the support structure 22 and are thus distributed around the circumference of the tubular body 11 to emit the lateral ultrasound waves in all directions, in particular in all 360 degrees around the circumference of the tubular body 11 away from the tubular body 11.

FIG. 3 shows a simplified schematic view of cross section through another catheter 110 according to the invention, wherein the cross section is positioned along the tubular body 111 at a position within the lateral ultrasonic sensor region 121 within the distal region of the tubular body 111. This catheter 110 is in most parts identical to the catheter 10 shown in FIG. 1. However, in the catheter 110, the support structure 122 on which the ultrasonic sensors 120.1 are mounted has a triangular cross section and surrounds the drainage lumen 112 having a circular cross section and running along the tubular body 111.

The invention is not limited to the embodiments illustrated in the context of the figures. Other variants and variations are readily available to the person skilled in the art.

In summary, it is to be noted that a catheter, in particular an external ventricular drainage catheter, for placement in a ventricular system, in particular of a human, is created for drainage of liquid, in particular of cerebrospinal fluid, from a ventricle of the ventricular system, pertaining to the technical field initially mentioned, that enables a reliable and safe use of the catheter for drainage of cerebrospinal fluid.

1. A catheter, for placement in a ventricular system, for drainage of liquid, from a ventricle of the ventricular system, said catheter comprising:
a) a tubular body having an internal drainage lumen running along said tubular body for the drainage of the liquid, and
b) at least one port arranged in a distal region of said tubular body, said at least one port connecting said drainage lumen with an outside of said tubular body and outside of said catheter for the drainage of the liquid from the ventricle into said drainage lumen,
wherein said catheter, further comprises at least one frontal ultrasonic sensor for emitting frontal ultrasound waves and detecting the frontal ultrasound waves reflected back to said at least one frontal ultrasonic sensor, wherein said at least one frontal ultrasonic sensor is arranged at a distal tip of said tubular body.

2. The catheter, according to claim 1, wherein said at least one frontal ultrasonic sensor is aligned to emit the frontal ultrasound waves from said distal tip of said tubular body away from said tubular body in a direction along a longitudinal axis of said distal region of said tubular body.

3. The catheter according to claim 1, wherein said at least one frontal ultrasonic sensor is adapted to emit the frontal ultrasound waves at a frequency in a range from 1 MHz to 20 MHz.

4. The catheter according to claim 1, wherein said at least one port comprises at least five ports arranged in said distal region of said tubular body, said at least five ports connecting said drainage lumen in said distal region of said tubular body with said outside of said tubular body and said outside of said catheter for the drainage of the liquid from the ventricle into said drainage lumen.

5. The catheter according to claim 1, wherein said at least one port is arranged proximally to said at least one frontal ultrasonic sensor.

6. The catheter claim 1, wherein said catheter further comprises at least one lateral ultrasonic sensor, for emitting lateral ultrasound waves and detecting the lateral ultrasound waves reflected back to said at least one lateral ultrasonic sensor, wherein said at least one lateral ultrasonic sensor is arranged laterally in said distal region of said tubular body.

7. The catheter according to claim 6, wherein said at least one lateral ultrasonic sensor is aligned to emit the lateral ultrasound waves away from said tubular body essentially in a direction in which said distal tip of said tubular body points.

8. The catheter, according to claim 6, wherein at least one lateral ultrasonic sensor is adapted to emit the lateral ultrasound waves at a frequency in a range from 1 MHz to 5 MHz.

9. The catheter according to claim 6, wherein said at least one lateral ultrasonic sensor comprises at least 3 lateral ultrasonic sensors for emitting lateral ultrasound waves and detecting the lateral ultrasound waves reflected back to said at least 3 lateral ultrasonic sensors, wherein said at least three lateral ultrasonic sensors are laterally arranged in said distal region of said tubular body.

10. The catheter according to claim 6, wherein said at least one port is arranged distally from said at least one lateral ultrasonic sensor.

11. The catheter according to claim 10, wherein each one of said at least one port is arranged distally from said at least one lateral ultrasonic sensor.

12. The catheter according to claim 1, wherein the tubular body comprises an antimicrobial coating.

13. The catheter according to claim 6, wherein said catheter includes a wiring connecting said at least one frontal ultrasonic sensor and said at least one lateral ultrasonic sensor with a connector for being connected to a control unit for controlling said at least one frontal ultrasonic sensor and said at least one lateral ultrasonic sensor, said connector being arranged in a proximal region of said catheter.

14. (canceled)

15. (canceled)

16. The catheter according to claim 1, wherein the catheter is an external ventricular drainage catheter.

17. The catheter according to claim 16, wherein the catheter is configured for drainage of cerebrospinal fluid from a ventricle of the ventricular system.

18. The catheter according to claim 1, wherein said at least one frontal ultrasonic sensor is at least one frontal ultrasonic transducer.

19. The catheter according to claim 3, wherein said at least one frontal ultrasonic sensor is adapted to emit the frontal ultrasound waves at a frequency in a range from 5 MHz to 12 MHz.

20. The catheter claim 6, wherein said at least one lateral ultrasonic sensor is at least one lateral ultrasonic transducer.

21. The catheter according to claim 8, wherein said at least one lateral ultrasonic sensor is adapted to emit the lateral ultrasound waves at a frequency in a range from 5 MHz to 12 MHz.
